(19) United States
(12) Patent Application Publication
(10) Pub. No.: US 2023/0355137 A1
(43) Pub. Date: Nov. 9, 2023

(54) COMPACT WIRELESS RANGE OF MOTION MEASUREMENT SYSTEM AND METHOD

(71) Applicant: X-ray Optics, Inc., Chicago, IL (US)

(72) Inventors: Ali KHOUNSARY, Chicago, IL (US); Jaymin Harshadkumar PATEL, Chicago, IL (US); David BORNEMAN, Chicago, IL (US); Gabriel BRYK, Chicago, IL (US)

(73) Assignee: X-ray Optics, Inc., Chicago, IL (US)

(21) Appl. No.: 17/735,835

(22) Filed: May 3, 2022

(51) Int. Cl. A61B 5/11; A61B 5/00

(52) U.S. Cl. CPC A61B 5/1122; A61B 5/6803; A61B 5/4566; A61B 5/7475; A61B 2560/0223; A61B 2562/0219; A61B 2562/0223

(57) ABSTRACT

Disclosed here are methods, systems, and devices for measuring cervical range of motion. Exemplary devices include wearable motion sensing devices integrated into eyeglass frames. The disclosure may measure cervical motion in six directions or any desired planes including Cartesian planes containing pitch, yaw, and roll movements to diagnose and track a user's recovery from a head and neck injury or ailment.

### TECHNICAL FIELD

The disclosure relates to the field of motion sensing devices. In particular disclosed herein are electronic motion sensing devices for monitoring patient health and recovery from injury.

### BACKGROUND

Musculoskeletal injuries are quite frequent, and often result in pain, constrained mobility or flexibility, and weakness. For example, whiplash is a very common musculoskeletal injury with about 3 million incidents per year in the US. Musculoskeletal injuries often require a long recovery time. Patients typically regain range of motion over a period of weeks or months when the injury has healed enough to allow movement with a tolerable amount of pain.

Despite the frequency of musculoskeletal injuries, measuring recovery progress is difficult. Healthcare providers have no objective way of simply, accurately, and easily measure the range of motion. Instead, providers rely on a patient's estimation of pain to gauge recovery progress. Pain can be subjective, inconsistent, and difficult to quantify, therefore, monitoring patient recovery using primarily pain estimation is problematic. Additionally, providers have no objective set of data other than their own memory to reference to come up with a good range of motion target for a given patient of a given age, gender, etc. With no available database of historical movement data, providers cannot predict how long, how many sessions, and or what exercises or treatment would be required for each patient to recover. Providers are also unable to track patient process and provide patient specific feedback on the patient's recovery progress. To design better recovery therapies that help patient recover faster, systems of accurately gauging recovery progress are needed. These systems should produce complementary recovery metrics that are more consistent than estimated pain and provide more accurate diagnosis and recovery time estimates for particular injuries. These systems should measure components of movement individually so that injuries in particular muscles and muscle groups can be identified.

### BRIEF DESCRIPTION OF THE DRAWINGS

Various objectives, features, and advantages of the disclosed subject matter can be more fully appreciated with reference to the following detailed description of the disclosed subject matter when considered in connection with the following drawings, in which like reference numerals identify like elements.

FIGS. 1-3 illustrate an exemplary wearable device for measuring range of motion, according to embodiments of the disclosure.

FIG. 4 is a circuit diagram illustrating exemplary electrical components that may be found within a wearable device, according to embodiments of the disclosure.

FIGS. 5A-B illustrate an exemplary system for measuring range of motion, according to embodiments of the disclosure.

FIG. 6A illustrates an exemplary positioning device, according to embodiments of the disclosure.

FIGS. 6B-6C illustrate methods for measuring orientation of a wearable device, according to embodiments of the disclosure.

FIGS. 7A-7C illustrate exemplary GUIs for displaying motion measurements, according to embodiments of the disclosure.

FIG. 7D illustrates an exemplary spherical coordinate system, according to embodiments of the disclosure.

FIG. 8 illustrates an exemplary laser alignment system, according to embodiments of the disclosure.

FIG. 9 illustrates an exemplary alignment routing, according to embodiments of the disclosure.

FIG. 10 is a flow chart illustrating an exemplary method for determining range of motion, according to embodiments of the disclosure.

FIG. 11 is a flow chart illustrating an exemplary method of monitoring patient recovery based on range of motion, according to embodiments of the disclosure.

FIG. 12 is a block diagram illustrating an exemplary client device, according to embodiments of the disclosure.

### DETAILED DESCRIPTION

Reference will now be made in detail to embodiments of the disclosure, examples of which are illustrated in the accompanying drawings. In the following detailed description, numerous specific details are set forth in order to provide a sufficient understanding of the subject matter presented herein. But it will be apparent to one of ordinary skill in the art that the subject matter may be practiced without these specific details. Moreover, the particular embodiments described herein are provided by way of example and should not be used to limit the scope of the invention to these particular embodiments. In other instances, well-known data structures, timing protocols, software operations, procedures, and components have not been described in detail so as not to unnecessarily obscure aspects of the embodiments of the disclosure.

### Overview

Disclosed herein are devices, systems, and methods for measuring range of motion. Systems may include wearable devices for measuring cervical range of motion, that is, the range of motion of a person's back, spine, or neck areas. The wearable devices may be electronic and portable to enhance convenience. The wearable devices may generate metrics that are consistent, trackable over time, and provide an objective diagnosis and assessment of recovery progress. For example, the wearable devices may determine a patient's range of motion in degrees of angular motion relative to an axis. The wearable devices described herein may produce more accurate measurements of range of motion faster and more efficiently relative to manual methods and alternative devices. The range of motion measurements may be trackable over time to help determine recovery process and diagnose injuries or other conditions that may affect range of motion. The wearable devices may incorporate a system for measuring range of motion into an eye glass frame to provide a range of motion measurement device that is light, portable, familiar to patients, and easy to use. Range of motion measurements as well as the trajectory (path) of the motions recorded/calculated by the wearable device may be used to track patient recovery progress over time and diagnose injuries and other conditions that effect range of motion. Range of motion measurements may be stored on the device or other computer system and analyzed to track the user's recovery process and analyze the movement data to estimate remaining recovery time, gauge the effectiveness of physical therapy treatments, prescribe user specific physical therapy regimes that facilitate faster recovery, and the like.

Range of motion data generated by the wearable device may be used to design physical therapy sessions that may facilitate faster recovery from injury. For example, the range of motion data may indicate a patient's range of motion along one axis (e.g., lateral bending around the roll axis of rotation) is not returning as fast as a patient's range of motion along another axis. Based on this data, patients may be prescribed physical therapy routines that specifically target improving lateral bending range of motion. Range of motion data generated by the wearable device may also be used to estimate recovery time for patients based on the patient's current and/or expected rate of improvement for range of motion. Range of motion data may also be used to diagnose an injury or determine an injury's severity. The range of motion data for each patient may be stored in a database and compared with other patient treatment and recovery records. The range of motion data may also be used as training data for one or more machine learning models that may diagnose injuries and other conditions affecting range of motion, predict a patient's rate of recovery, generate an anticipated recovery timeline, predict a patient's range of motion after a treatment, and the like.

### Movement Measurement Device Hardware

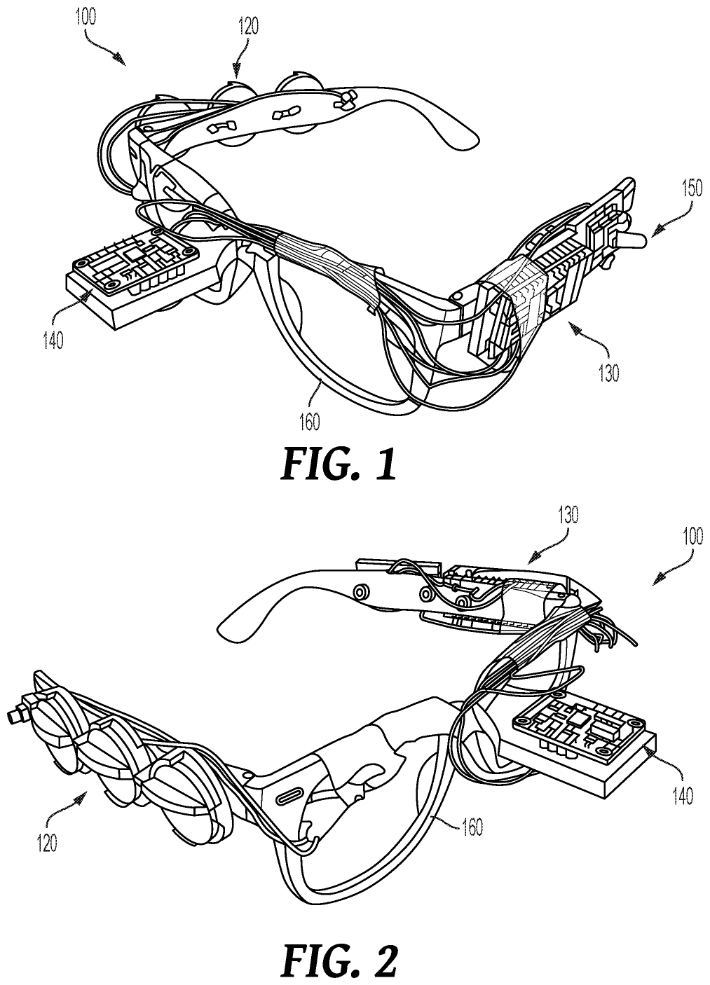

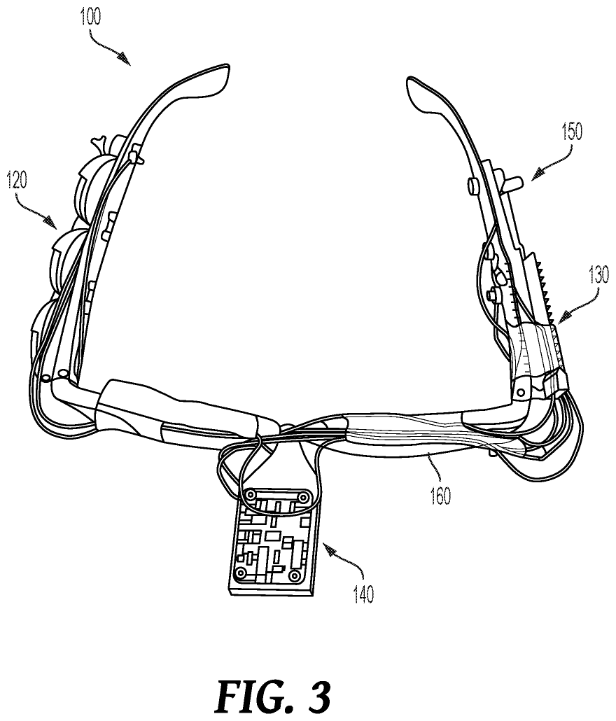

FIGS. 1-3 illustrate an exemplary wearable device 100 for measuring cervical range of motion. The wearable device 100 may be worn by a user, in accordance with an embodiment of the present disclosure. In various embodiments, the wearable device 100 may be configured to be worn over the user's ears and rest on the bridge of the user's nose and ears similar to a pair of glasses. FIG. 1 illustrates a right isometric view of the wearable device 100, FIG. 2 illustrates a left isometric view of the device, and FIG. 3 illustrates a top view of the wearable device 100. In some example embodiments, prescription lenses and/or tinted lenses may be placed into the frame 160 of the device such that they may act as normal glasses or sunglasses as well as the range of motion system described herein for continuous tracking and measurement of range of motion.

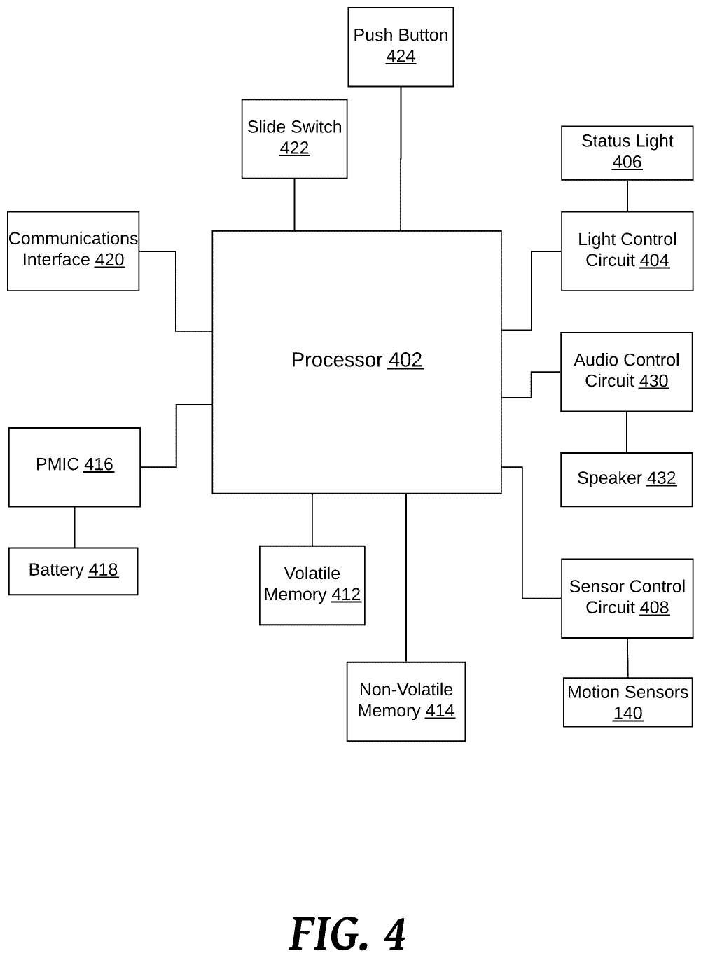

The wearable device 100 may integrate one or more electronics components into an eyeglass frame 160. The one or more electronics components may be integrated into the eyeglass frame at the center of the nose bridge, into one or two temples, or both. The one or more electronics components may extend out laterally from the nose bridge so that the electronics components are positioned adjacent to the temples of the wearer. The electronics components may include one or more movement measurement devices 140 that measure movement data (e.g., angles, movement trajectories, or other measurements that describe range of motion in one or more planes during one or more movements) and a microprocessor 130 that receives movement data (e.g., cervical range of motion measurements) from the movement measurement devices 140 and transfers the data via wire or wirelessly to a portable device or a computer. The wearable device 100 may also include a power source 120 including a charging mechanism for powering the electronics components and an on/off switch 150 to actuating and powering down the wearable device 100. The charging mechanism may be a wired and or wireless charging mechanism. The wearable device 100 may use movement data obtained from one or more movement measurement devices 140 to measure a user's cervical range of motion. FIG. 4 illustrates a circuit diagram of an exemplary wearable device 100. As shown, the wearable device 100 may include a printed circuit board (PCB) including various electronics components. In various embodiments, the PCB may include a processor 402 for executing commands and instructions of one or more of the components. Suitable processors 402 for the execution of a program of instructions may include, by way of example, microcontrollers or microprocessors and the sole processor or one of multiple processors or cores of any kind of computer. For example, the processor 402 may be an Atmega328 microcontroller manufactured by Atmel®. Generally, a processor 402 may receive instructions and data from a volatile memory 412 or a non-volatile memory 414 or both. Suitable volatile memory 412 may include RAM, high speed memory, double data rate memory, 4R memory, and the like. Suitable non-volatile memory 414 may include a MultiMediaCard (MMC), embedded MultiMediaCard (eMMC), solid-state drive (SSD), and the like.

The processor 402 may be coupled to one or more control circuits. For example, the processor 402 may be coupled to a sensor control circuit 408 to control movement measurement devices 140 used to measure a patient's cervical range of motion. The movement measurement devices 140 may include one or more sensors, for example, an accelerometer, magnetometer, and/or gyroscope or sets thereof. In some examples, these sensors may be configured to sense motion in one, two, and/or three directions/dimensions/planes. Movement data collected by the sensors may be in the form of an acceleration vector describing angular motion along an axis of rotation (e.g., pitch, yaw, roll) in three-dimensional space. The computing systems may combine these measurements in order to calculate more complex movements such as rotation, acceleration, motion trajectory, etc. The sensor control circuit 408 may control one or more aspects of the movement measurement devices 140, for example, the sample rate of the movement measurement devices 140, execution of one or more calibration processes, the component of the patient's cervical range of motion measured by the movement measurement devices 140 (e.g., the axis of rotation for each measurement), and the like. To improve the accuracy and reliability of movement data, the sensor control circuit 408 may also include logic that initiates collection of movement data by the movement measurement devices 140 and transmission of movement data to the communications interface 420. For example, the sensor control circuit 408 may ensure movement data measurements are collected and/or transmitted only after the movement measurement devices 140 have be calibrated.

In various embodiments, the movement measurement devices 140 provide precise tracking of a particular type of movement (e.g., cervical motion in various planes). When integrated into an eyeglass frame, the movement measurement devices 140 provide a distinctly different device, construct, purpose, function, protocol, and application than head mounted displays (HMD), and a simpler, less expensive, lighter weight, and disposable alternative to other more elaborate hardware that measures head movement for other applications. HMDs include robust sensor arrays that require a tremendous amount of processing capacity and electric power to operate. These systems are expensive and much heavier than a pair of eyeglasses. Additionally, HMDs are configured to track a general position of a user's head. Conversely, the movement measurement devices 140 are devised and developed to be positioned and oriented on a patient's head such that they allow consistent measurements and tracking head movement with high accuracy, precision, and repeatability. The data processing required to track head movement using the movement measurement devices 140 is specialized and optimized and the devices use the minimum measurement number of specialized sensors in an efficient manner. The movement measurement devices 140 also precisely positioned and oriented precisely for each patient before every measurement session. The initial positioning and orientation alignment techniques described below enable the movement measurement devices 140 to provide head movement and tracking data in a repeatable manner over many measurement sessions with accuracy and precision compared with r other motion tracking devices.

The processor 402 may interface with a communications interface 420 to facilitate communicating with external devices. For example, the communications interface 420 can include a wireless computer and communications module for connecting to an external device (e.g., a laptop, an external hard drive, a tablet, a smart phone) for transmitting the data and/or messages to the external device. The communication interface 420 can include cellular transmission capability allowing the device to be used in telemedicine or other real time communications applications. For example, the communications interface may use cellular communication to transmit real time data to a server and further transfer data including head motion trajectory plots to severs, personal computers, or other computer systems accessible by attending medical practitioners. In various embodiments, the wireless communications module may include a Wi-Fi chip, an embedded Bluetooth module, and the like. The communications interface 420 may transmit data using any known wired or wireless communications protocol, for example, Bluetooth, Wi-Fi, and the like.

A power management integrated circuit (PMIC) 416 may be integrated into the PCB and is responsible for controlling a battery charging circuit to charge a battery 418 or other power source 120. In various embodiments, the PCB may include built-in LiPoly charger that interfaces with a USB controller to charge the battery 418 by plugging a wall charger into a USB port coupled to the processor 402. The battery 418 supplies electrical energy for running the electrical components of the wearable device. The PCB can further include a slide switch 422 and a push button 424 for operating the wearable device. For example, a user may turn on or off the device by pressing the push button 424. The PMIC 416 may also have automatic a system shut off switch (e.g., a digital switch) that automatically powers down the wearable device 100 after a predetermined period of time (e.g., 5 minutes, 10 minutes, or any other timer period). The automatic system shut off switch may be programmed by the user to power down the wearable device 100 after any predetermined period of time and the duration of the period of time may be determined by the user and may be variable depending on one or more properties of the battery 418 (e.g., current charge level) and or wearable device 100. The PMIC 416 may also include an automatic system turn on switch (e.g., a digital switch) that automatically turns on the wearable device 100 in response to an event (e.g., a movement of the device that is detected by the movement sensing devices 140). The user may switch between different modes (e.g., motion capture, calibration, continuous capture) using the slide switch 422 and/or push button 424. The processor 402 can further control a light control circuit 404 for controlling the status lights 406. The status lights 406 can include, e.g., multiple light-emitting diodes (LEDs) in different colors for showing various status of the wearable device.

The PCB may also include an audio control circuit 430 that may control a speaker 432. For example, the audio control circuit 430 may operate the speaker 432 to generate a sound in response to a particular event (e.g., a patient moving outside of a particular plane while performing a movement during a range of motion measurement session. The audio control circuit 430 may also receive audio inputs from a microphone 434. The audio control circuit 430 may generate control signals from the audio inputs to enable users to control the wearable device 100 using verbal commands. The communications interface 420 may also communicate with a client device of other device having a speaker that is connected to the wearable device to generate the sound.

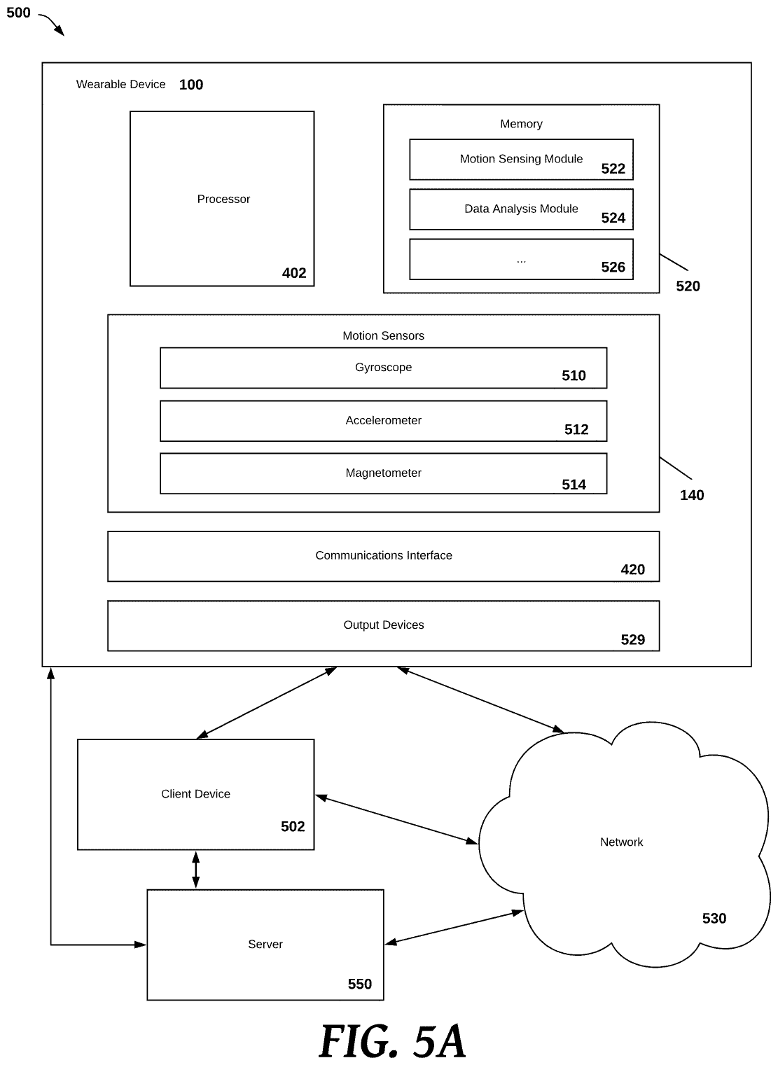

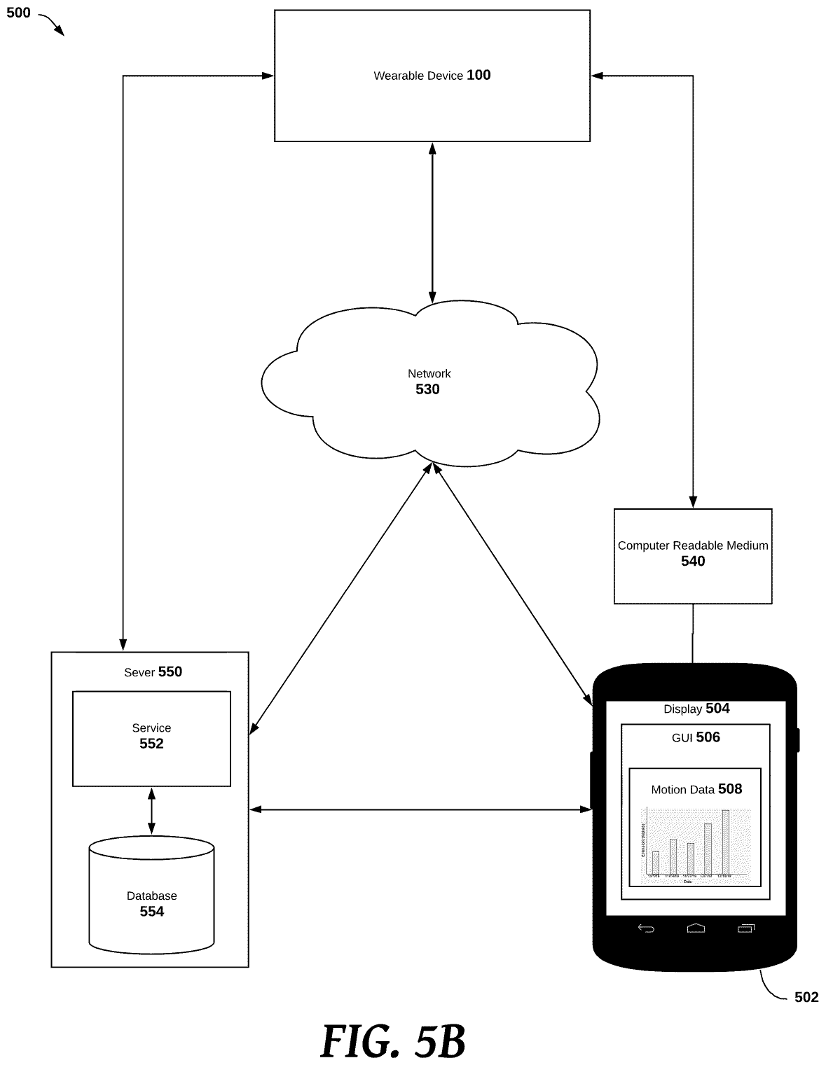

FIGS. 5A-B depict an exemplary system for measuring cervical range of motion and analyzing cervical range of movement data. The system may include a plurality of functional elements that may be provided by mechanical components, electrical components, and/or computing devices. These elements may work together to measure a user's cervical range of motion along multiple planes of motion and in some examples, display the results to the user, and/or send data to a back-end system for analysis or storage.

FIG. 5A includes a block diagram of the wearable device 100 illustrating exemplary components that may be found within the wearable device 100. The wearable device 100 may be communicatively coupled to one or more client devices 502. The wearable device 100 and one or more client devices 502 may communicate directly through any wired and/or wireless communications protocol. For example, the wearable device 100 may communicate with one or more client devices 502 wirelessly via Bluetooth. The wearable device 100 may also communicate with one or more client devices through a network 530. For example, communication between the wearable device 100 and the client devices 502 may be facilitated by one or more application programming interfaces (APIs). APIs of the system may be proprietary and/or may be examples available to those of ordinary skill in the art such as Amazon® Web Services (AWS) APIs, Google APIs, and the like. Network 530 may be the Internet and/or other public or private networks or combinations thereof.

In some embodiments, the wearable device 100 can include a processor 402 (or “application processor” or “AP”), a memory 520, one or more movement measurement devices 140, and one or more output devices 529 (e.g., speakers for playing music or emitting sound notifications, microphones for receiving audio inputs that may be used as verbal commands for controlling the wearable device 100, lasers that may be used to align the movement measurement devices 140, displays, and the like). The wearable device 100 may include additional modules, fewer modules, or any other suitable combination of modules that perform any suitable operation or combination of operations.

### Cervical Motion Measurement Device Component Communications

FIG. 5B includes a block diagram illustrating communications between components that may be included in the exemplary system for measuring cervical range of motion and analyzing movement data 500. As shown in FIG. 5B, the wearable device may be coupled to client device 502 and a server 550. The wearable device 100, server 550, and or client device 502 may be configured to communicate with one another through network 530. For example, communication between the elements may be facilitated by one or more application programming interfaces (APIs). APIs of system 500 may be proprietary and/or may be examples available to those of ordinary skill in the art such as Amazon® Web Services (AWS) APIs or the like. Network 530 may be the Internet and/or other public or private networks or combinations thereof.

The server 550 may be configured to implement a service 552, which may be used to input movement data via network 530 from one or more databases 554, the wearable device 100, and or client device(s) 502. The server 550 may execute processing instructions provided by the service 552 to receive, transmit, store, and/or analyze movement data. For example, the server 550 may receive cervical range of motion data measured by the wearable device 100 and store the received movement data in one or more databases 554. The one or more database 554 may store a complete record of all movement data captured by the wearable device 100 and or all movement data captured for a particular user and thereby correlated in the database to an identifier for particular users and/or user accounts. The server 550 may analyze movement data to generate charts and other insights from the movement data. For example, the server 550 may generate a chart for tracking a user's progress in recovering from injury, compare a user's movement data to movement data from other users or other external data (e.g., motion data collected by the wearable device manufacturer that is segmented by age or other characteristics i.e., motion data for healthy females aged 20-25), and or provide a record of the user's recovery process over a set time period (e.g., two weeks, a month, and the like). The server 550 may transmit the chart and or other movement data to the client device 502 for display to the user. Although only one instance of the server 550, client device 502, and wearable device 100 is shown in the example, it is understood that embodiments of the system 500 include more than one server 550, client device 502, and a wearable device. For example, one or more servers 550 may receive movement data from two or more wearable devices 100 and one or more servers 550 may transmit charts and other movement data to two or more client devices for display to plurality of users. The one or more servers 550 may store the movement data in a secure location (i.e., a medical records system database). The one or more servers 550 may also transmit the movement data to other applications and or devices for further analysis.

### Software Overview

As described, the hardware of the system is able to calculate, record, and cause storage of data from the various movement measurement devices. Once that data is calculated and sent for processing, the systems and methods here may be used to analyze that data, cause display of that data, and/or visual representations of what that data means for a person or account for a person, for further analysis and physical therapy feedback. Movement data may be used to track a patient's progress while recovering from an injury. For example, movement data collected by the device may be displayed relative to healthy patients and/or the patient's own range or motion before the injury to demonstrate the difference between the patient's current cervical range of motion and the patient's anticipated range of motion when fully recovered. Patients may use the device to measure range of motion consistently throughout the recovery process. The record of movement data collected by the device may be used to estimate the patient's remaining recovery time and help develop physical therapy treatments that facilitate faster recovery.

As shown in FIG. 5A, the wearable device may include one or more processor(s) 402 configured to run various software applications and modules. Software including the movement sensing module 522, data analysis module 524, and an alignment module 526 can run on any processor 402 capable of executing computer instructions or computer code. The processor 402 can also be implemented in hardware using an application specific integrated circuit (ASIC), programmable logic array (PLA), field programmable gate array (FPGA), or any other integrated circuit.

Memory 520 can include volatile memory 412 and non-volatile memory 414 as described above. The one or more software modules 522-526 may be stored in memory along with data generated by the modules 522-526. The processor 402 can be configured to run one or more modules 522-526 that are configured to cause the processor 402 to perform various steps that are discussed throughout the present disclosure. To track movement data for multiple patients that may use the same wearable device 100, the one or more software modules 522-526 may each create a patient profile that may store patient data (e.g., name, address, contact information, data of birth, height, gender, weight, race, injury history, physician, insurance data, and the like). The patient data may be manually entered on a client device 502 connected to the wearable device 100 and or automatically populated from an electronic medical records system or other application that may interface with one or more software modules 522-526 of the wearable device 100.

The processor 402 may receive and process data from one or more movement measurement devices 410. The movement measurement devices 140 can include one or more sensors (e.g., a gyroscope 510, an accelerometer 512, and/or a magnetometer 514). The wearable device 100 may include multiple movement measurement devices 140 to increase accuracy and reliability of the movement data measured by the wearable device 100. For example, the wearable device 100 may include multiple movement measurement devices 140 mounted at different angles to generate multiple pieces of movement data that may be combined or otherwise manipulated to increase accuracy. For example, the multiple pieces of movement data may be averaged and or compared to reject outlier measurements generated by one or more uncalibrated, broken, and or malfunctioning measurement devices.

The accelerometer 512 may be a three-axis accelerometer that measures linear acceleration in up to three-dimensions (for example, x-axis, y-axis, and z-axis). The gyroscope 510 may be a three-axis gyroscope that measures rotational data, such as rotational movement and/or angular velocity, in up to three-dimensions (for example, yaw, pitch, and roll). For example, the accelerometer 512 may be a triaxial 14-bit accelerometer, the gyroscope 510 may be a close-loop triaxial 16-bit gyroscope, and the magnetometer 514 may be a triaxial geomagnetic sensor. The processor 402 may receive movement data and/or information from one or more movement measurement devices 140 to track acceleration, rotation, position, and or orientation information of the wearable device 100 in six degrees of freedom through three-dimensional space.

Before each movement measurement session, the movement measurement devices 140 may be aligned using the alignment module 526 to ensure consistent measurements are made. Cervical movement data collected by the movement sensing module 522 may be transmitted to the data analysis module 524 for further analysis. After receiving movement data collected during a measurement session, the data analysis module 524 may execute one or more operations to refine movement data before it is transmitted to a client device 502. For example, the data analysis module 524 may time stamp movement data points, average a series of motion measurements, organize movement data measurements by axis of motion (e.g., pitch (extension, flexion), roll (lateral bending), or yaw (rotation) and/or motion component (e.g., left rotation, right flexion, left bending, and the like), convert the movement data to another form or unit, and/or perform other statistical operations to remove outliners, improve data quality, and/or facilitate analysis of movement data. The data analysis module 524 may also generate one or more graphs, plots, charts, and/or other data visualizations that may be displayed on the client device 502 and/or a display integrated into the wearable device 100. The data visualizations may be assembled based on movement data (i.e., angles of motion relative to the roll, pitch, and yaw axes) recorded by the movement measurement devices. The data visualizations may include 2D and or 3D models that illustrate a trajectory of motion of the patient's head and or neck during a series of movements. For example, the data visualizations generated when the patient rotates his head to the right and left may show the patient moving out of the horizontal plane appreciably once or more during that left and right rotational motion (i.e., the patient may go out of plane at 35-degree rotation and back into plane at 39 degrees rotation during the motion). The trajectory of motion shown by the data visualizations can help patients, healthcare providers, and other users determine and track recovery progress and or diagnose injuries or other conditions affecting range of motion.

### Cervical Movement Measurement Device Initial Positioning, Orientation, and Calibration

To generate movement data, the wearable device may determine the number of degrees of rotation the patient deviates from a set initially calibrated at-rest position. The motion sensing module may set an initial position of the wearable device as a fixed frame of reference for measuring motion. Before determining an initial position, the wearable device may be calibrated. Calibration may include verifying the wearable device is connected to a client device 502, network 530 and or server 550. Calibration may also include confirming the movement measurement devices are capturing measurements within an acceptable range of error. The movement measurement devices may be calibrated using a calibration function (e.g., an auto-calibration function installed on the wearable device and or client device). If the wearable device is not connected to a client device, an on-board recording apparatus, and/or one or more of the movement measurement devices is malfunctioning, the measurement session may not be initialized.

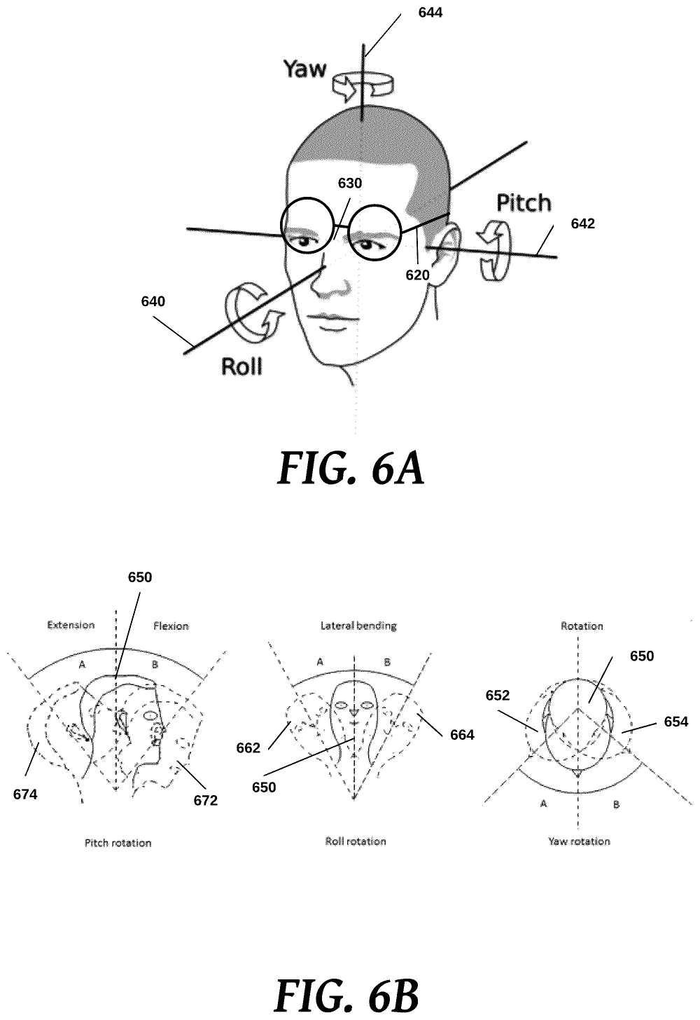

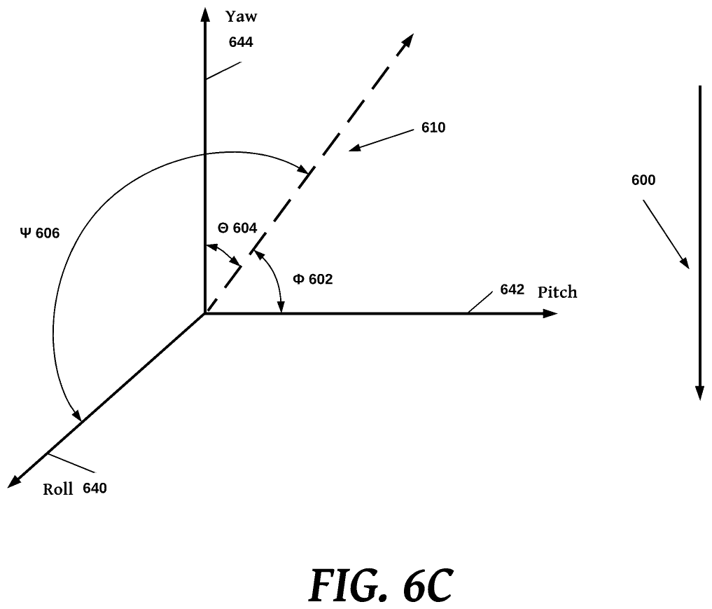

In various embodiments, the wearable device may include hardware for positioning the wearable device at the same location on a patient's face during each measurement session. FIG. 6A illustrates an example positioning clip 620 that may be integrated into one or more temples 624 of the eyeglass frame. To ensure consistent measurements over many sessions and visits to potentially different health care providers, i.e., measurement repeatability, that initial position and the orientation of the wearable device on each patient's face must remain unchanged. To position the wearable device at the same location on a patient's face every time, the eyeglass frame is pressed against the face until the bridge meets the top of the nose. The positioning clip 620 is then moved and tightened in place over the ear of the patient. For example, the positioning clip 620 may be tightened into place using a screw knob 628 included in the positioning clip 620. The screw knob 628 may secure the positioning clip 620 on the eye glass frame using a screw that pushes against the temple 624 when the screw knob 628 is rotated downward (e.g., clockwise). The screw knob 628 may be rotated upward (e.g., counterclockwise) to release the positioning clip 620 from the temple 624. A bottom side of the positioning clip 620 may include a curved edge 622 that is ergonomically shaped to match the top edge of the patient's ear. The curved edge 622 may be positioned to fit over the ear of the patient at the same location every time and secured with the screw knob 628 to ensure that the wearable device 100 is positioned precisely at the same location on the head consistently every time.

Once the frame bridge is tightly positioned against the nose bridge and the positioning clip 620 is resting fit on the patient's ear and secured to the temple 624, the position of the positioning clip 620 may be recorded. For example, the position of the positioning clip 620 relative to one or more of the calibrated positions 626 on the temple 624 may be recorded. To ensure the repeatability and accuracy of the measurements collected by the wearable device over time, in different settings, and by different operators, all measurements for each patient may be taken with the wearable device in the same initial position every time. The initial position of the wearable device on each patient may be located by fitting the frame and the positioning clip 620 to the patient as describe above. Locating the initial position of the wearable device for each patient before each measurement session ensures the wearable device is always placed the same way with respect the patient's head. Orientation consistently is ensured through components 800 and 900 described below. Additionally, the adjustable nature of the positioning clip 620 may allow the wearable device to have a one size fits all frame.

The position clip 620 aligns the wearable device and patient's head with the local Cartesian coordinates to ensure consistent initial positioning. The patient's head and wearable device are then moved as a rigid combination to align the patient's head with a local external reference. For example, the alignment of the patient's head may be achieved using lasers integrated into the wearable device as described below. The initial positioning and alignment of the wearable device ensures consistent measurements by placing the wearable device on a patient's head in a consistent manner, and also by orienting the patient's head and the wearable device to the same initial orientation for every measurement session.

### Movement Data Collection and Analysis

The wearable device 100 may measure a patient's cervical range of motion by determining movement data for a patient as the patient performs a pre-determined set of movements. The pre-determined set of movements may include movements in three orthogonal planes (i.e., moving the patient's head to the left and or right around the roll axis, moving the patient's head forward and or backward around the pitch axis, and rotating the patient's head to the right and or left around the yaw axis.) The pre-determined set of movements may also include freestyle movements in which the patient can move his head in any direction he feels most comfortable. The movement measurement devices included in the wearable device may measure movement data in three-dimensions (i.e., in three orthogonal planes along three axes of rotation). FIGS. 6B-C illustrate the three axes of rotation and movements measured by the wearable device. To measure the patient's range of motion, the movement measurement devices of the wearable device may receive data from the individual single planar movement measurement devices and combine the data to extrapolate complex, multi-dimensional movement such as acceleration vectors, yaw, pitch, and roll, etc. FIG. 6B illustrates the three axes of rotation including the roll axis 640, pitch axis 642, and yaw axis 644. FIG. 6C illustrates movements of a user's head in three orthogonal planes along each axis of rotation. A center position 650 of the user's head is shown in solid lines and the rotation positions are shown in dashed lines.

The movement measurement devices may measure movement data continuously throughout the pre-determined set of movements made by the patient so that healthcare providers can track the patient's complete range of motion and determine the manner in which the patient completes each movement. For example, the continuous tracking of the patient's head during the pre-determined set of movements may be used to determine if the patient's movement is consistent (i.e., has a constant rate of motion) or uneven (i.e., has different rates of motion at different positions). A consistent movement may be an indicator of healthy, strong muscles and pain free range of motion. Alternatively, uneven movement may be an indicator of weak muscles and painful movement.

The movement measurement devices enable the wearable device to monitor the patient's movement in three orthogonal planes continuously throughout the full range of motion. To more precisely measure the patient's movement within one plane, the wearable device may make in-plane measurements. The in-plane measurements are used to determine the patient's range of motion within one plane (i.e., the patient's right to left movement within the pitch roll plane of FIG. 6C). If the patient moves his head outside of the plane used for the in-plane measurement at or beyond a pre-determined angle and or distance (e.g., 3 degrees or any other angle and or distance), the wearable device may emit an audio sound to alert the patient that he has moved outside of the plane. The loudness of the audio sound may correspond to the angle and or distance outside of the plane the patient moves. For example, the loudness of audio sounds for movements 5 degrees outside of the plane may be greater than the loudness of audio sounds for movements 3 degrees outside of the plane. The audio sound may continue until the patient moves back inside the plane for the in-plane measurement. The audio feedback provided to the patient ensures consistency in the movement measurements over several treatment sessions.

The audio sound feedback provided by the wearable device regulates the movement of the patients and thereby enables the movement measurement devices to take more accurate in-plane measurements. Additionally, in comparison to the freestyle movements typically used in clinical settings, the in-plane measurements provide a more consistent and complete set of movement data. Practitioners may use the in-plane measurements to track the patient's recovery in three orthogonal planes to help monitor and restore the patient's full range of motion. The in-plane measurements may also be used by practitioners to identify specific components (i.e., extension, flexion, right/left lateral bending, right left rotation, and the like) of the patient's range of motion that are deficient and administer treatments and therapies that specifically target the deficient range of motion components.

As shown in the FIG. 6C, the yaw axis of rotation 644 describes rotating the head to the left and to the right from center. For example, rotation around the yaw axis of rotation 644 includes rotating the head to a left position 652 to the left of the center position 650 and rotating the head to a right position 654 to the right of the center position 650. The roll axis of rotation 640 describes lateral bending or right and left flexion movement of the head that occurs when the top of head is tilted to the left and the right from center. For example, rotation around the roll axis of rotation 640 includes laterally flexing the head to a left tilted position 662 to the left of the center position 650 and laterally flexing the head to a right tilted position 664 to the right of the center position 650. The pitch axis of rotation 642 describes extension or forward and back flexion movement of the head that occurs when the head is moved forward and backward from center. For example, extending the head back to backward tilted position 674 behind the center position 650 and flexing the head forward to a forward tilted position 672 in front of the center position 650.

FIG. 6C illustrates an exemplary motion along each axis of rotation related to a fixed frame of reference 600 with respect to the initial position of the wearable device 100. For example, the initial position of the wearable device 100 may be set with the user's head in the center position 650 as shown in FIG. 6B. In this orientation, the fixed frame of reference has the yaw axis of rotation 644 perpendicular to the top surface of the movement measurement devices of wearable device 100. The pitch axis of rotation 642 and the roll axis of rotation 640 for the fixed frame of reference 600 can be chosen relatively arbitrarily as long as the three axes are perpendicular to each other. As shown in FIGS. 6B and 6C, the pitch axis of rotation 642 is parallel with the direction of the bridge 643 of the eyeglass frames and the roll axis of rotation 640 is parallel with the direction of the one or more temples 624 of the eyeglass frames.

As shown in FIG. 6C, movement of the user may be measured relative to the frame of reference 600 by the various arrangements of movement measurement devices described herein (See examples in FIGS. 1-3). For example, when the user moves to a device position 610, the device measures an angle (ϕ) 602 with respect to the positive pitch axis of rotation 642, an angle (θ) 604 with respect to the positive yaw axis of rotation 644, and an angle (ψ) 606 with respect to the positive roll axis of rotation 640. Users may operate the device to measure only a single component of their cervical range of motion. For example, users may configure the wearable device to measure an angle that describes at least one of the extensions of the user's neck, the flexion of a user's neck, the left rotation of the user's neck, the right rotation of the user's neck, the left flexion of the user's neck, and the right flexion of the user's neck. The wearable device may also be configured to measure all components of a user's cervical range of motion (i.e., angle values in three orthogonal directions) simultaneously with one function.

To move the wearable device to device position 610, the user rotates their head to the right to produce an angle (θ) 604 with respect to the positive yaw axis of rotation 644 that is less than 90°. The various movement measurement devices may generate data regarding the device position with an angle (θ) with respect to the positive yaw axis of rotation 644 that is more than 90° as reflecting a rotation of the user's head to the left from center. To move the wearable device to device position 610, the user extends their head forward to produce an angle (ϕ) 602 with respect to the positive pitch axis of rotation 642 that is less than 90°. The various movement measurement devices may generate data regarding the device position with an angle (ϕ) with respect to the positive pitch axis of rotation 642 that is more than 90° as reflecting a user extending their head back from center. To move the wearable device to device position 610, the user tilts their head laterally to the left to produce an angle (ψ) 606 with respect to the positive roll axis of rotation 640 that is more than 90° which may be sensed by the various movement measurement devices to generate data regarding the rotation. The various movement measurement devices may generate data regarding the device position with an angle (ψ) that is less than 90° as reflecting a user tilting their head laterally to the right from center.

The wearable device may be configured to measure cervical range of motion during a specific set of movements performed by the patient. For example, the wearable device may measure a left rotation of the user's neck while the user isolates their neck and gradually rotates to the left. Movement measurements captured by the wearable device may include angles of motion as well as one or more trajectories of motion that illustrate the path of a patient's head movement. The motion sensing module may indicate specific movements the user may perform to generate each measurement. For example, the motion sensing module may play a description of the movement (e.g., rotate head to the right, tilt head back, etc.) over a speaker built into the wearable device or included in a remote client device. The wearable device may also be configured to measure the user's cervical range of motion during any activity including normal, everyday activities. For example, the wearable device may measure cervical range of motion based on movements performed while the user is driving, walking, or watching television.

The motion sensing module may also store range of motion measurements collected over time. The historical movement data may be used to track the patient's range of motion throughout the recovery process to help track the patient's rate of recovery. The historical movement data may also be used to determine specific therapies and treatments that are beneficial to increasing the patient's range of motion and specific therapies and treatments that have not had an impact and or reduced the patient's range of motion. The movement data for a patient may also be compared to historical movement data for healthy and or injured individuals to help diagnose injuries and or determine how the patient's characteristics (e.g., age, gender, race, weight, injury history) impact recovery and disability as a result of injury.

### Exemplary GUIs for Movement Data

Client devices 502 may be any device having a display 504 configured to present graphical user interfaces (GUIs) 506 including one or more pieces of movement data 508 and receive inputs thereto in the GUIs 504. For example, client devices 502 may be smartphones, personal computers, tablets, laptop computers, wearables such as the glasses themselves, watches, or other devices. The client devices 502 may include voice and video conferencing functionality that enables telemedicine. For example, the movement data 508 may be automatically shared with a physician or other healthcare provider and or uploaded to a patient record in an electronic medical records system accessible by the healthcare provider to enable patients and healthcare providers to review the movement data 508 during a telemedicine appointment and or other live remote consultation. As part of the telemedicine appointment, the patient may measure his range of motion in real time during the live consultation with the remote healthcare provider. To help the patient use the wearable device, the healthcare provider may give instructions to the patient on how to sit, how to position the wearable device on the patient, and or how to move. The movement data collected by the movement measurement devices during the movements performed by the patient may be sent to the healthcare provider in real time so that the healthcare provider may review the data and give the patient feedback based on the movement data. The movement data 508, processing instructions (e.g., instructions for reading movement data, instructions for manipulating movement data, instructions for generating the GUIs 506, and the like) and components of the GUIs (e.g., tables, charts, formatting instructions) may be stored in a computer readable medium 540. The computer readable medium 540 may be any computer readable memory or storage.

The display 504 may be a screen, such as a crystalline (e.g., sapphire) or glass touchscreen, configured to provide output to the user as well as receive input from the user via touch. The GUIs 506 output on the display 504 may receive input from the user to select, for example, which movement data should be displayed, or whether the user is starting a movement data measurement session, ending a measurement session, or calibrating the wearable device 100. In some embodiments, wearable device 100 may present output to the user in other ways, such as by producing sound with a speaker, and wearable device 100 may receive input from the user in other ways, such as by receiving voice commands via a microphone. Inputs received from users and other data generated by the client device may be stored in the computer readable medium 540.

Movement data collected by the wearable device may be transmitted to a client device and displayed in a GUI rendered on a display of the client device. Movement data may be displayed in a GUI after it is collected by the wearable device or in real time during collected. To facilitate accurate measurements, an animated visualization of one or movements required for measuring cervical range of motion data may also be displayed in a GUI on the remote client device during a measurement session.

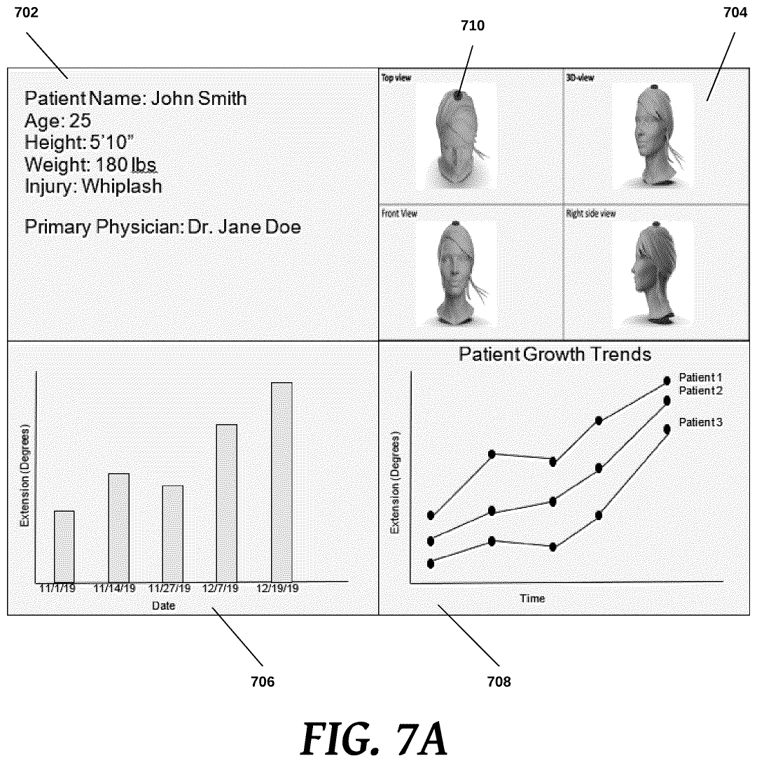

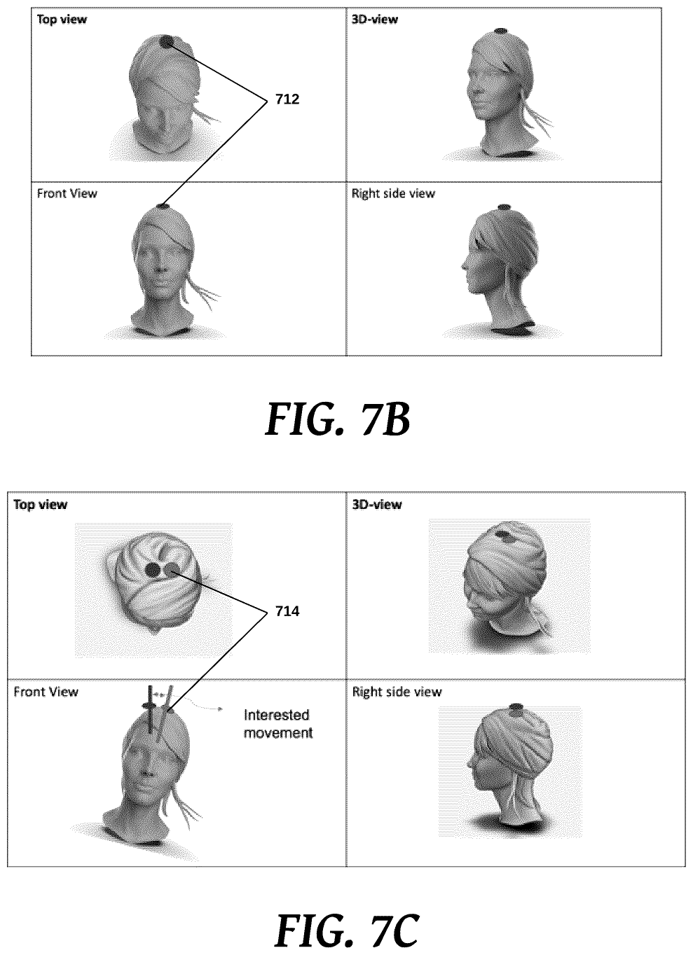

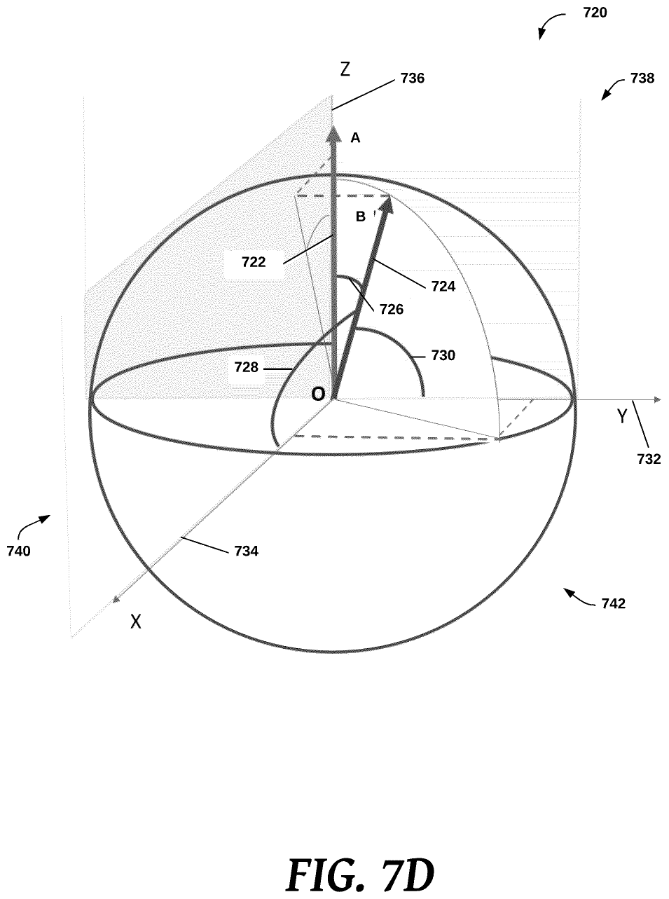

FIGS. 7A-C illustrate exemplary GUIs generated by the wearable device. FIG. 7A illustrates an exemplary four panel display with each panel displaying different information. The upper left panel 702 includes user information and injury information. The top right panel 704 includes a 3D model visualizing the user's range of motion. The 3D model may position a first dot (e.g., a red dot) 710 at the user's initial position. A second dot may show the user's range of motion relative to the initial position. The 3D model may be generated by displaying the angles measured by the movement measurement devices during the movements performed by the patient in a spherical coordinate system (e.g., the spherical coordinate system shown below in FIG. 7D). The angles measured by the movement measurement devices may also be projected onto three orthogonal planes to generate 2D visualizations for the patient's range of motion (e.g., the 2D visualizations of in-plane movements shown in FIG. 6C). The 3D models can depict the trajectory and or path of head movement in 3-D to illustrate the patient's general head movement. The 2D visualizations can depict the trajectory and or path of head movement in any direction on any plane including on a set of three orthogonal planes in real time to illustrate the patient's head movement in a particular direction.

The lower panels in FIG. 7A may illustrate graphs showing the user's range of motion in a particular direction over time. For example, the lower left panel 706 shows the user's degrees of extension over an 8-week recovery period. The bars illustrate a user's extension range of motion (in degrees) at measurement sessions taken every two weeks over the 8-week period. As shown in the graph, the user gradually regained range of motion over the 8-week period. The GUIs generated by the wearable device may also illustrate the user's progress relative to healthy patients or other benchmarks. For example, the lower right panel 708 illustrates the user's extension range of motion over time during a period of recovery from injury. The user data may be stored such that it is correlated with users or patient identifiers. In such a way, various users or patients may have their data stored, analyzed, and graphed for various feedback (e.g., injury diagnosis, recovery projection, and the like) and further analysis. One such example shows data from “Patient 1” and the line graph may illustrate the rate of recovery for the user relative to “Patient 2” and “Patient 3”.

FIGS. 7B-C illustrate close up of the 3D model shown in the upper right panel 704 of the four-panel display shown in FIG. 7A. FIG. 7B illustrates a 3D model with a first set of dots (e.g., red dots illustrating an initial position for the user at the beginning of a measurement session) 712. FIG. 7C illustrates a 3D model showing the user's range of motion. The 3D model includes a second set of dots (e.g., green dots) 714 illustrating the range of motion measured for a user during a measurement session. The second set of dots 714 may show the maximum movement from the initial position the user was able to achieve during a measurement session.

The wearable device may calculate the degrees separating the maximum movement from the initial position and may display the degrees of movement to the user. The degrees of movement may be expressed within a spherical coordinate system 720. For example, the spherical coordinate system 720 shown in FIG. 7D. The green dot shown in FIG. 7C may correspond to a head direction at an initial position shown in the spherical coordinate system 720 as vector A 722. The initial position of the patient's head (represented by vector A 722) may go through a pivot point in the neck (e.g., a central pivot at the center of the neck) that corresponds to the origin (i.e., “0”) of the spherical coordinate system 720. Therefore, the initial position of the patient's head may be represented by Vector A 722 that runs long the Z direction centered at “0” in the spherical coordinate system 720 that is fixed in space.

During a measurement period, the patient may perform a freestyle movement (i.e., an arbitrary compound rotation) to move his head to a first position represented by vector B 724. To determine, the patient's range of motion during the freestyle movement, the movement measurement devices may measure the angles between the first position (i.e., vector B 724) and the x axis 734, y axis 732, and z axis 736. The pitch angle may correspond to the angle between vector B 724 and the y axis, the roll angle may correspond to the angle between vector B 724 and the x axis, and the yaw angle may correspond to the angle between the vector B 724 and the z axis. The movement measurement devices may also measure the angles between vector B 724 and the xy plane, the xz plane, and the yz plane to determine the range of motion of the patient at the first position. As described above, the degrees of movement measured by the movement measurement devices may be displayed to the user along with a visualization (e.g., a 2D or 3D visualization) that tracks the patient's movement. The 2D and 3D visualizations may include an animation that shows the patient's movement in real time.

As described above, the movement measurement devices provide three sets of values including roll, pitch, and yaw angle measurements. For example, the three sets of values include starting angles, ending angles, and real time angle values around the roll, pitch, and yaw axes measured during movements performed by the patient. The angle values around the roll, pitch, and yaw axes may be compiled to generate one or more trajectories of motion that illustrate the path of the patient's head and or neck movement.

As the patient's head is moved, the device generates plus or minus (depending on the direction) values for roll and pitch and a number between 0 and 360 degrees for the yaw. For example, for right/left flexion (measured by the angle of roll rotation) right flexion is positive while left flexion is denoted negative. Similarly, for extension/flexion (measured by the angle of pitch rotation), the extension value is positive, and the flexion value is negative. The rotation of the neck in the horizontal plane (measured by the angle of yaw rotation) is measured clockwise from 0 to 360 degrees. For example, 45 degrees clockwise rotation shows as 45 while 45 degrees rotation in the counterclockwise shows as 315.

During a measurement, the patient's initial position (i.e., the position that corresponds to the red dot in FIG. 7C) is recorded and optionally set to zero (i.e., 0,0,0). All measurements during the particular measurement session are made relative to this initial position. The patient is asked to move his head in a series of specific directions to perform the set of pre-determined movements required to generate a full set of cervical range of motion data. The movement measurement devices continuously measure the angles during each movement. The data analysis module calculates from the raw movement data the trajectory, velocity, the maximum angle, and other statistics that describe the patient's motion. The statistics calculated by the data analysis module may be displayed, for example, as the bar chart shown in the lower left panel 706 of FIG. 7A.

Table 1 below includes exemplary recorded movement data measured by the movement measurement devices of the wearable device for in-plane motions performed during a measurement session. All movement data is shown in degrees. The patient's initial position is displayed is (3.45, 2.21, 1.03), but it could also be set to zero to make the movement data easier to interpret.

TABLE 1

| Description | Raw Sensor Output | Displayed Output |
|---|---|---|
| Initial (starting) position of the head | (3.45, 2.21, 1.03) | |
| Move head extension to read 55.29 | (3.45, 55.29, 1.03) | |
| Extension amount | (3.45, 55.29, 1.03) | (55.29 − 2.21) = 53.08 |
| Move head Flexion to read −45.86 | (3.45, −45.86, 1.03) | |
| Flexion amount | | −(−45.86 − 2.21) = 48.07 |
| Rotation (yaw) to the right to read 76.34 | (3.45, 2.21, 76.34) | |
| Rotation (yaw) to the right amount | | (76.34 − 1.03) = 75.31 |
| Rotation (yaw) to the left to read 288.66 | (3.45, 2.21, 288.66) | |
| Rotation (yaw) to the left amount | | (360 − 283.66 + 1.03) = 77.37 |
| Right Lateral Flexion (roll) to read 44.83 | (44.83, 2.21, 1.03) | |
| Right lateral flexion (roll) amount | | (44.83 − 3.45) = 41.38 |
| Left lateral flexion (roll) to read −55.29 | (55.29, 2.21, 3.45) | |
| Left lateral flexion (roll) amount | | (−55.29 − 3.45) = 58.74 |

As shown in Table 1, the initial position of the patient is (3.45, 2.21, 1.03). During the measurement session, the patient performs an extension motion (i.e., flexes his neck to move his head vertically backward), the pitch angle is increased by the motion from 2.21 degrees to 55.29 degrees. All other angles remain constant because the exertion motion was an in-plane motion that restricted movement to just one plane. Based on the difference in the pitch angle measured during the motion and the pitch angle measured at the initial position of the patient, the motion sensing module records a maximum extension of 53.08 degrees. Similarly, when the patient performs his next motion (i.e., a flexion motion that requires the patient to flex his neck to move his head vertically forward) the pitch angle is decreased from 2.21 degrees to −45.86 degrees. Based on the difference between the pitch angle measured at the initial position and the pitch angle measured during the motion, the motion sensing module records a maximum flexion of 48.07 degrees. The process is repeated for the other in-plane motions (right and left rotation and right and left lateral flexion) to obtain a complete set of cervical range of motion data.

The wearable device may display angles of movement for all 6 in-plane motions to the user by generating a separate 2D/3D model for each motion (e.g., a first model for right rotation, a second model for left flexion, and the like) and/or a single 3D model with different sets of dots showing motion in each direction. The wearable device may also change the perspective of the 3D model to emphasis a particular motion. The wearable device may also place an additional set of dots illustrating the expected range of motion for the user once they fully recovery from the injury.

The data analysis module may also present a line graph of other visualization that shows the patient's movement data over time (i.e., historical movement data). For example, the lower right panel 708 of FIG. 7A displays collected movement data (i.e., extension range of motion) for the patient over time. As shown in the chart, the patient's range of motion (i.e., degrees of extension) increases overtime. The data analysis module may calculate statistics from the historical movement data that describe the patient's improvement. For example, the data analysis module may calculate the patient's rate of improvement, the patient's average improvement across all measurement sessions, the patient's percent increase or decrease in range of motion at a measurement session relative to a pervious measurement or baseline minimum or maximum value, and the like. The data analysis module may also compare the patient's movement data to stored data for other healthy and or injured patients. The data analysis module may retrieve the movement data from other patients from a local and or cloud database and may display the movement data from other patients alongside the patient's movement data to provide context about the patient's progress. For example, the chart shown in the lower right panel 708 of FIG. 7A shows the patient's (i.e., patient 1) extension range of motion data and the extension range of motion data for two other patients (i.e., patient 2 and patient 3). The comparisons between the patient's movement data and the stored movement data from the other patients may illustrate patient's current status and or recovery rate relative to prior patients' progress.

The data analysis module may also train one or more machine learning models using the patient's data and or stored movement data from other patients. The machine learning models may generate predictions related to the patient's recovery. For example, the machine learning models may estimate the patient's recovery time, predict the patient's rate of recovery, predict the patient's maximum range of motion after recovery, and the like. The data analysis module may also use one or more algorithms or heuristics to extrapolate recovery time, rate of recovery, maximum range of motion, and other recovery metrics based on trends included in the patient's data and or trends included in stored data from other patients.

### Correcting for Errors in Movement Data

The movement measurement devices are precise and accurately measure the angles in three orthogonal planes generated movements performed by the user. Despite the precision and accuracy of the movement measurement devices, range of motion measurements for patients may still be undermined by the placement of the wearable device on the patient and the patient's posture when performing the movements used to measure the movement data. To maintain the reliability and accuracy of movement data captured over time, it is important that each measurement session begin with the patient's head held in the same initial position and orientation. The wearable device may be placed on the same position on the patient's body during each measurement session to enhance the reliability and accuracy of movement data. Without mitigation, slight variation in the patient's position (e.g., where the patient sits, the patient's posture, how high the patient is off the ground, if the patient moves during the measurement session, and the like) and the position of the wearable device (e.g., how high the frame sits on the bridge of the patient's nose, how horizontally flat the frame sits of the patient's face, how far laterally to the right and or left from center are the movement measurement device located, and the like) can disturb the alignment of the movement measurement devices and reduce the reliability and accuracy of the movement data.

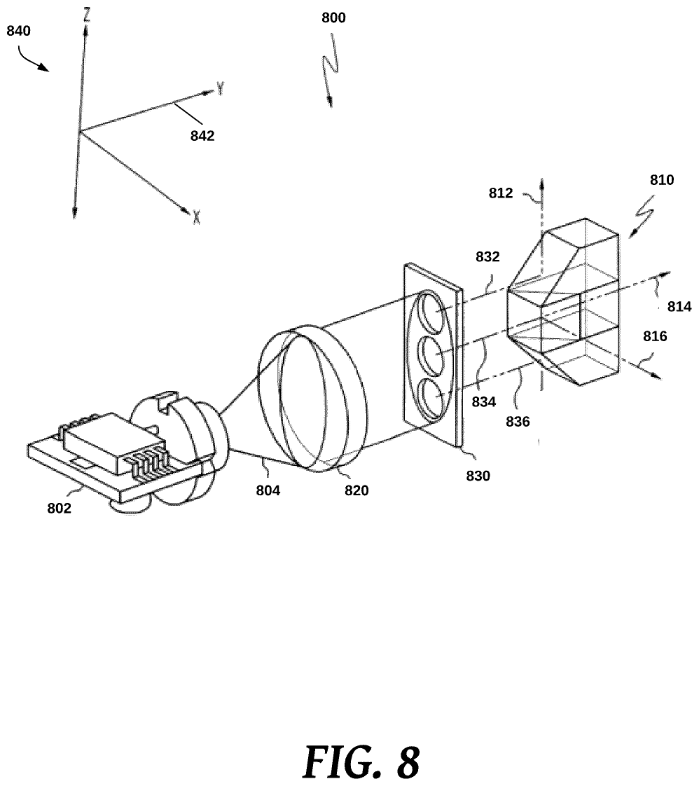

To ensure the position of the patient and the wearable device is in the same during every measurement period, provisions to place the wearable device on the face at the same location every time and hold the head in the same orientation every time are provided. The wearable device may include a laser alignment system 800. FIG. 8 illustrates and exemplary laser alignment system 800 that may be mounted on one side of the frame of the wearable device. For example, the laser alignment system 800 may be mounted on the frame approximate to the frame temples (i.e., the portion of the frame that aligns with the temples on the head of the patient). The laser alignment system may include a single light source 802 generating a beam of light 804 that shines on an optic 810. The optic 810 may produce three orthogonal beams 812, 814, and 816 which are oriented and aligned so as to appear to be originating from a single coincident point within the optic 810. The light source 802 is preferably a laser diode which generates a beam of light 804 which is generally elliptical in cross-section. The beam of light 804 is projected generally along the y-axis 842 of the coordinate system 840. The beam of light 804 (i.e., the elliptical laser beam) passes through a collimating lens 820 and a beam splitter 830. The beam splitter 830 may include one or more apertures of any size and or shape (i.e., rectangular apertures, round apertures, slits, crosses, and the like). For example, the beam splitter 830 may include a horizontal slit at the center hole and vertical slits at the top and bottom holes. As the collimated beam of light may passes through the beam splitter 830, the beam splitter produces three parallel, collimated beams of laser light 832, 834, and 836. Beam 834 (i.e., the “level” beam) has approximately half the power of the sum of the apertured beam 804. Beams 832 and 836, (i.e., the “Plumb” beams), each have approximately a quarter of the power of the sum of the apertured beam 804. The three beams 832, 834, and 836 are projected onto the optic 810. The optic 810 both reflects and transmits the three beams 832, 834, and 836 to produce three orthogonal beams 812, 814, and 816 of about equal power. The three orthogonal beams 812, 814, and 816 may be oriented and aligned to appear to be originating from a single coincident point within the optic 810. The orthogonal beams 812, 814, and 816 may have a line profile that may be visible on a surface (e.g., a wall, piece of ceiling, mirror, and the like). The three orthogonal beams may be projected in various directions. For example, the top beam 812 may be reflected up towards the ceiling of a room, the middle beam 814 may be projected forward on to a wall directly in front of the middle beam, and the lower beam 816 may be reflected to the right towards a wall to the right of the middle beam 814.

The optic 810 may be comprised of optical components. Each optical component may have a simple geometric shape. The optical components can be comprised of borosilicate crown glass such as, for example, BK7 glass. The optical components may preferably have an optical surface flatness of a quarter wavelength at 635 nm.

In various embodiments, fewer orthogonal lines may be used to align the movement measurement devices. For example, the laser alignment system 800 may not require a beam splitter 830 and may have a simplified optic 810 including only one horizontal slit that produces a bright horizontal beam of light. The horizontal beam of light may be projected on to a wall. To align the movement measurement device, the patient may line up the horizontal beam of light with a horizontal line on the wall. The horizontal line on the wall is precisely and consistently positioned to ensure proper alignment. For example, the horizontal line is drawn at a consistent predetermined distance up from the floor. The horizontal line must also be level (i.e., fit between two parallel lines) to serve as an effective reference for alignment. Once the position of the horizontal line is set, any deviation of the horizontal beam of light (i.e., a position up or down from the horizontal line, a position to the right or left of the horizontal line, and or if the horizontal beam of light is rotated relative to the horizontal line) indicates the wearable device is not properly positioned. To fix the alignment of the movement measurement device, the patient adjusts the position of the wearable device and or the position of patient's head until the horizontal beam of light aligns with the horizontal line on the wall.

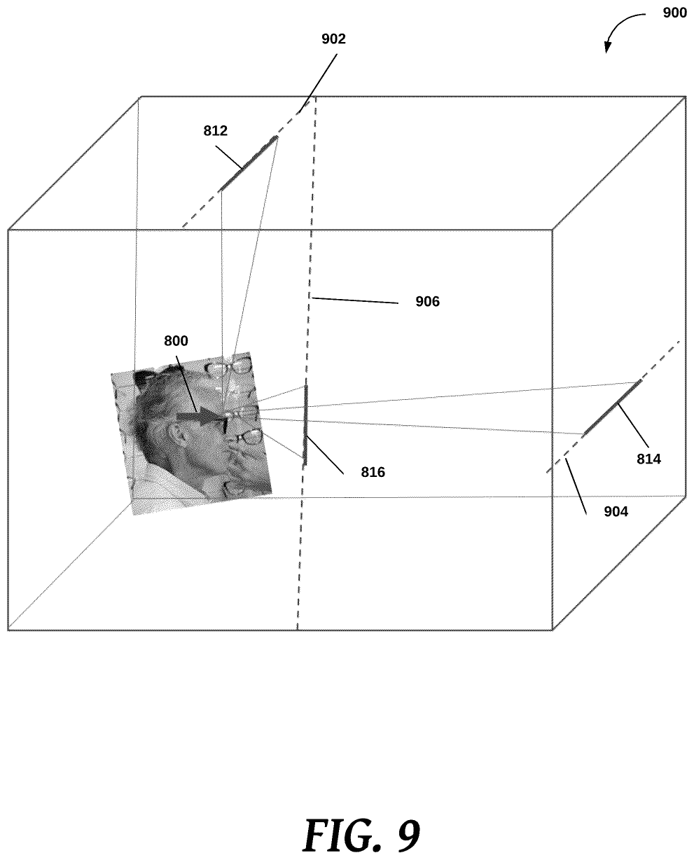

The laser alignment system 800 may be used to align the movement measurement devices of the wearable device in the same position during every measurement period. FIG. 9 illustrates an exemplary alignment routine that uses the laser alignment system 800. The frame design of the wearable device ensures the placement of the glasses on the patient's nose and ears are reasonably repeatable for each patient. Additionally, the support provided by the patient's nose and ears hold the wearable device nearly horizontal when worn on the patient's face. Therefore, it can be assumed that the placement of the wearable device is relatively constant throughout all measurement sessions.

To ensure the initial position of the patient's head is consistent throughout all measurement sessions (i.e., the patient's position and posture are the same) the laser alignment system 800 may be used to align the patient's head at a consistent initial position within an examination room 900. The aid in the alignment, the examination room may include one or more marked lines. FIG. 9 illustrates three marked lines but as few as one marked line may be used to align the movement measurement devices. In the illustrated embodiment with three marked lines, the top marked line 902 may be positioned on the ceiling of the examination room, the forward marked line 904 may be positioned on the wall of the examination room directly in front of the patient, and the side marked line 906 may be positioned on the wall of the examination room that is to the left and or right of the patient. The patient's posture and or position may be adjusted (i.e., the patient's chair height and or orientation may be adjusted) until the orthogonal beams 812, 814, and 816 generated by the laser alignment system align with (i.e., touches and or overlaps) the marked lines 902, 904, and 906 on the walls of the examination room. For example, the top orthogonal beam 812 aligns with the top marked line 902, the straight orthogonal beam 814 aligns with the forward marked line 904, and the side orthogonal beam 816 aligns with the side marked line 906. The marked lines 902, 904, and 906 may be visible lines that are physically drawn or otherwise marked on the walls of the examination room 900. The marked lines 902, 904, and 906 may also be imaginary lines and or temporary lines that are not physically marked on the walls of the examination room 900. Once the orthogonal beams 812, 814, and 816 are aligned with their corresponding marked lines 902, 904, and 906, the angles of the patient's position are read by the movement measurement devices. The angles at the patient's position are then zeroed to set the patient's initial position for the measurement session. To determine the patient's range of motion, the movement measurement devices measure the angles relative to the initial position in three orthogonal planes as the patient's performs the pre-determined set of movements.

### Exemplary Method of Measuring Cervical Range of Motion

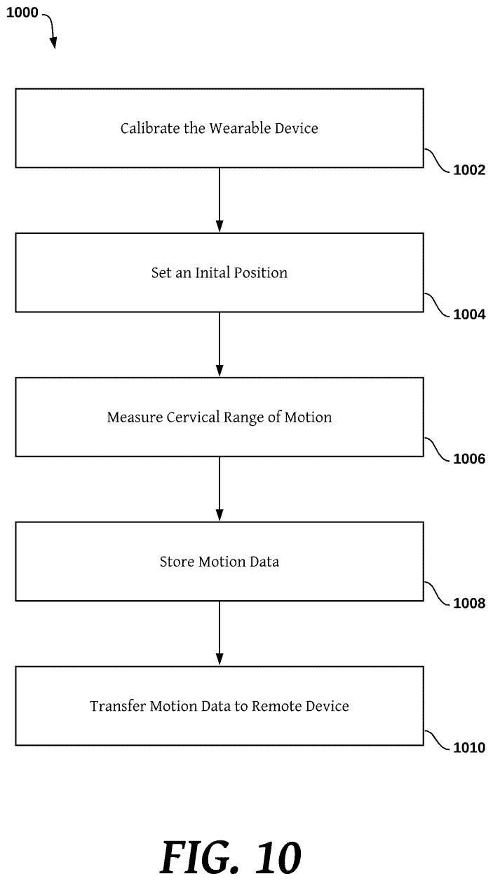

FIG. 10 is a flow chart illustrating an exemplary method of determining a patient's range of motion using the wearable device 1000. To determine a patient's range of motion, the patient first puts on the wearable device. For example, the patient may put on a pair of glass frames incorporating the electronics components of the wearable device. At 1002, the wearable device is calibrated. Device calibration may be initialized automatically upon powering on the wearable device and/or manually initialized by sliding a switch or pressing a push button. Device calibration may include verifying the wearable device is connected to a remote device and/or calibrating the movement measurement devices to ensure they are capturing movement data within a predefined error range. After calibration, the wearable device may initialize a range of motion measurement session by setting an initial position for the range of motion measurements at 1004. The initial position may be set automatically upon detecting the user's head is still by the movement measurement devices. The initial position may also be set manually in response to a user actuating a slide switch or push button.

At 1005, the movement measurement device is aligned to ensure the initial position is consistent across different measurement sessions. For example, the movement measurement device may be aligned using the laser alignment system described above. At 1006, the wearable device measures and collects data regarding the user's range of motion relative to the initial position set at 1004 from the movement measurement devices. The wearable device may measure the user's range of motion by determining all six directions of the user's cervical range of motion simultaneously. The wearable device may also measure one or more components of the user's range of motion individually. For example, the wearable device may measure a left rotation of the user's neck while the user performs an isolated left rotation of their neck. The wearable device may then measure the right rotation of the user's neck while the user performs an isolated right rotation of their neck. The remaining components of cervical range of motion including forward and backward extension of the neck and right and left lateral flexion of the neck may also be measured individually while the user performs isolated movements for each motion. The wearable device may also measure one or more components of the user's cervical range of motion during any motion performed by the user including motion during everyday activities such as walking or driving.

Communication of the data may be made to a back-end system, wireless computer such as a smartphone or laptop for storage and analysis. For example, the data may be transferred to an electronic medical records system and or shared with a physician or other healthcare provider one a secure, live video conferencing platform that provides for telemedicine consultations. In some examples, movement data measured by the wearable device may be stored remotely, locally, and/or in a distributed way at 1008. Movement data may be stored in memory or written to a database or other storage integrated into the wearable device. Movement data may also be transferred to a client device or other remote computer via any communication protocol (e.g., Bluetooth or other near field communication protocol) at 1010. Movement data transferred from the wearable device may then be stored on the remote computer. Movement data stored at 1008 may be used to track a user's cervical range of motion over time. For example, movement data may be used to monitor a user's range of motion while the user recovers from a musculoskeletal injury. Movement data collected by the wearable device may be analyzed to predict the length of the user's recovery period, the recovery time required to achieve a target cervical range of motion, and the user's cervical range of motion when fully recovered. Movement data collected by the wearable device may also be used to provide feedback to physicians, physical therapists, and other healthcare providers about the user's recovery. Movement data for each component of cervical range of motion may be used to prescribe physical therapy treatments that target specific components of range of motion that the user is not making enough process on during recovery. For example, movement data may be analyzed to determine the user needs more physical therapy treatments that target enhancing left neck rotation, forward neck extension, or some other component of cervical range of motion. Finally, a user interface may be used to cause display of data before or after analysis and computations are made. In such a way, charts, graphs, 3D models, or other displays may be computed and displayed for a user, caregiver, and/or healthcare provider for use.

### Exemplary Use Case for Cervical Motion Measurement Device

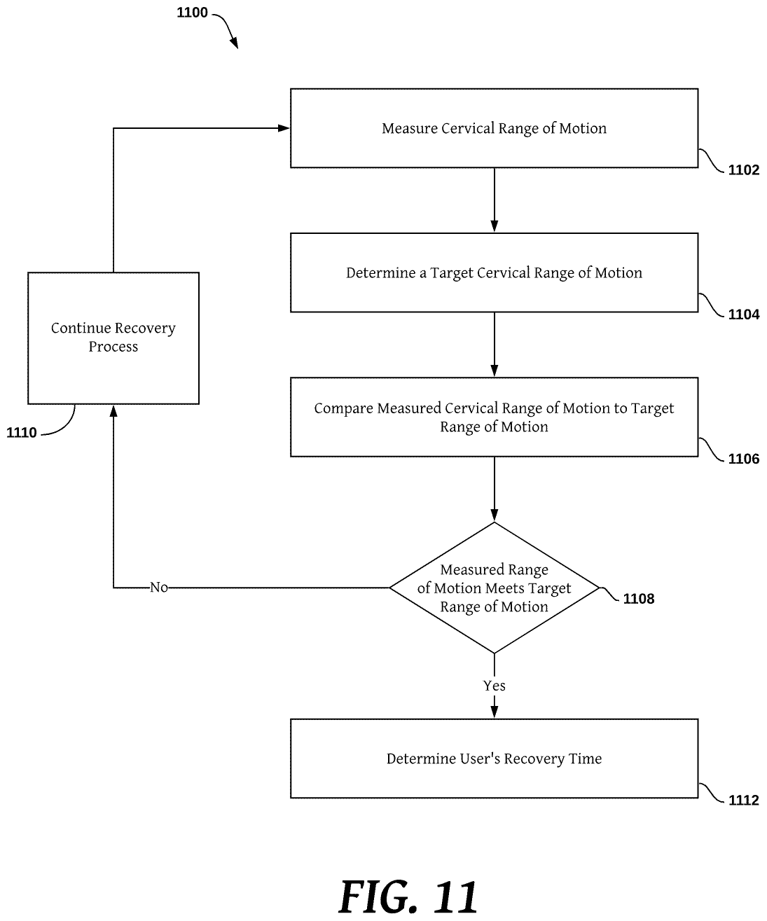

FIG. 11 is a flow chart illustrating an exemplary method for monitoring a user's recovery 1100. At 1102, the wearable device measures the user's range of motion as described above. At 1104, the wearable device determines a target cervical range of motion. The target cervical range of motion may be a desired range of motion the user (i.e., patient, physician, physical therapist, and the like) wants to achieve. The target cervical range of motion may be a specific target for a particular component of cervical range of motion or may be a general target for all components of cervical range of motion. A target cervical range of motion may be determined based on movement data for the user (e.g., movement data captured by the wearable device during previous measurement sessions) or movement data from other patients. For example, the target range of motion may be the expected range of motion of a healthy person or a person that is fully recovered from the user's injury. The target range of motion may also be the expected range of motion for a patient having the same characteristics as the user (e.g., age, gender, size, injury type, injury severity, and the like) during their recovery process. For example, the expected range of motion for a patient may be two weeks, a month, two months, and the like after suffering the injury. Movement data from other patients may be collected by an instance of the wearable device during previous measurement sessions and stored in a central database included cervical range of motion data for all measurement sessions performed by every instance of the wearable device.

The user's cervical range of motion measured by the wearable device may then be compared to the target range of motion at 1106. To facilitate a comparison, the wearable device may measure the user's range of motion in real time and generate a graph, chart, figure, or other visual representation of the user's range of motion relative to the target range of motion. For example, the wearable device may generate a line graph that tracks the user's range of motion over time against the range of motion of other patients. Range of motion measurements for each new measurement session may be plotted on the graph with the degrees of movement shown vertically on the y axis and the time since injury shown horizontally on the x axis.

If at 1108, the user's range of motion meets or exceeds the target range of motion the wearable device may determine the user's recovery time (i.e., the amount of time since the injury required for the user to achieve the target range of motion) at 1112. For example, if the user's degrees of motion surpass the degrees of motion for the target range of motion at or before the set recovery time for the target range of motion, the wearable device may determine the user's measured range of motion meets the target range of motion. If the user's measured range of motion does not meet the target range of motion (e.g., the patient has fewer degrees of motion than the target range of motion), the user may continue their recovery process at 1110. After continuing recovery for any period of time (e.g., one hour, one day, two weeks, and the like). The user may then remeasure their cervical range of motion at 1102 and repeat steps 1104-1106 to monitor the user's recovery. The wearable device may also request a description of the physical therapy treatments the users received in order to achieve or not achieve the target range of motion. The wearable device may then associate the physical therapy treatment with achieving or not achieving target range of motion outcomes to provide practitioners additional data about the effectiveness of physical therapy treatments for musculoskeletal injuries.

### Computer Device Examples

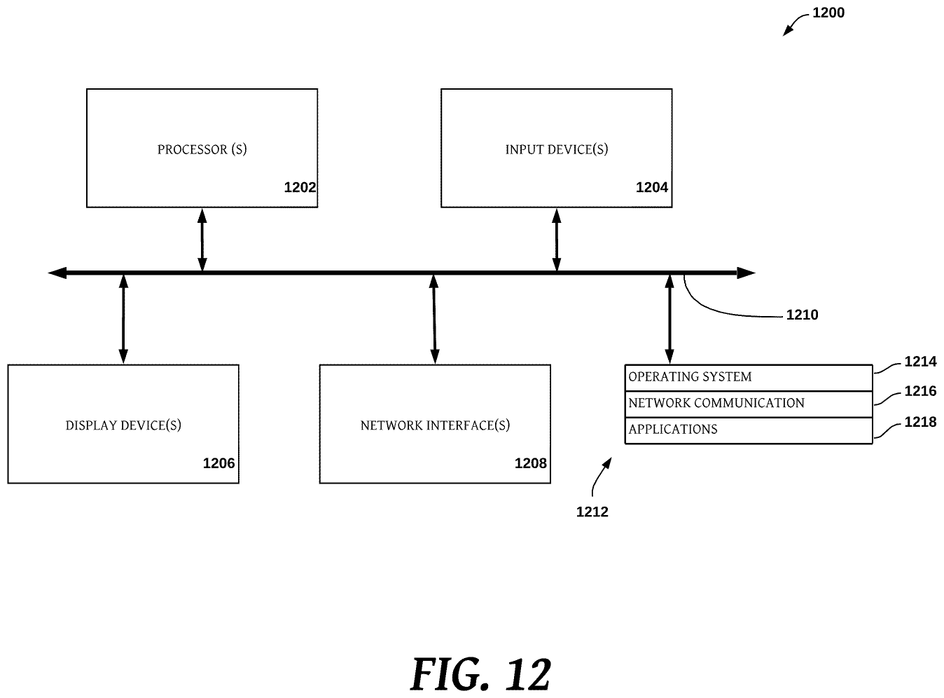

FIG. 12 shows more details of any computing system disclosed herein including but not limited to a client device 502 from FIG. 5B, the Wearable Device 100 of FIG. 5A, back-end server 550 of FIG. 5B, or any other computing system according to an embodiment of the present disclosure. The computing device 1200 may include a system for transmitting, storing, analyzing and/or displaying movement data. The computing device 1200 may be implemented on any electronic device that runs software applications derived from compiled instructions, including without limitation personal computers, servers, smart phones, media players, electronic tablets, game consoles, email devices, etc. In some implementations, the computing device may include one or more processors 1202, one or more input devices 1204, one or more display devices 1206, one or more network interfaces 1208, and one or more computer-readable mediums 1212. Each of these components may be coupled by bus 1210, and in some embodiments, these components may be distributed among multiple physical locations and coupled by a network.

Display device 1206 may be any known display technology, including but not limited to display devices using Liquid Crystal Display (LCD) or Light Emitting Diode (LED) technology. Processor(s) 1202 may use any known processor technology, including but not limited to graphics processors and multi-core processors. Input device 1204 may be any known input device technology, including but not limited to a keyboard (including a virtual keyboard), mouse, track ball, camera, and touch-sensitive pad or display. Bus 1210 may be any known internal or external bus technology, including but not limited to ISA, EISA, PCI, PCI Express, USB, Serial ATA or FireWire. Computer-readable medium 1212 may be any medium that participates in providing instructions to processor(s) 1202 for execution, including without limitation, non-volatile storage media (e.g., optical disks, magnetic disks, flash drives, etc.), or volatile media (e.g., SDRAM, ROM, etc.).

Computer-readable medium 1212 may include various instructions 1214 for implementing an operating system (e.g., Mac OS®, Windows®, Linux). The operating system may be multi-user, multiprocessing, multitasking, multithreading, real-time, and the like. The operating system may perform basic tasks, including but not limited to recognizing input from input device 1204; sending output to display device 1206; keeping track of files and directories on computer-readable medium 1212; controlling peripheral devices (e.g., disk drives, printers, etc.) which can be controlled directly or through an I/O controller; and managing traffic on bus 1210. Network communications instructions 1216 may establish and maintain network connections (e.g., software for implementing communication protocols, such as TCP/IP, HTTP, Ethernet, telephony, etc.).

Application(s) 1218 may be an application that uses or implements the processes described herein and/or other processes. For example, a data analysis application that generates visualizations, analyzes movement data to monitor user recovery, executes one or more operations to transform movement data to another unit, facilitates manual analysis of movement data and physical therapy treatments by physicians, and the like. The processes may also be implemented in operating system 1214. For example, application 1218 and/or operating system 1214 may present GUIs 506 including movement data 508 which may include results from data analysis tasks as described herein.

### CONCLUSION

The described features may be implemented in one or more computer programs that may be executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program may be written in any form of programming language (e.g., Objective-C, Java), including compiled or interpreted languages, and it may be deployed in any form, including as a stand-alone program or as a module, component, subroutine, or other unit suitable for use in a computing environment.

Suitable processors for the execution of a program of instructions may include, by way of example, microcontrollers, special purpose microprocessors, and the sole processor or one of multiple processors or cores, of any kind of computer. Generally, a processor may receive instructions and data from a read-only memory or a random-access memory or both. The essential elements of a computer may include a processor for executing instructions and one or more memories for storing instructions and data. Generally, a computer may also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory may be supplemented by, or incorporated in, ASICs (application-specific integrated circuits).

To provide for interaction with a user, the features may be implemented on a computer having a display device such as an LED or LCD monitor for displaying information to the user and a keyboard and a pointing device such as a mouse or a trackball by which the user can provide input to the computer.

The features may be implemented in a computer system that includes a back-end component, such as a data server, or that includes a middleware component, such as an application server or an Internet server, or that includes a front-end component, such as a client computer having a graphical user interface or an Internet browser, or any combination thereof. The components of the system may be connected by any form or medium of digital data communication such as a communication network. Examples of communication networks include, e.g., a telephone network, a LAN, a WAN, and the computers and networks forming the Internet.

The computer system may include clients and servers. A client and server may generally be remote from each other and may typically interact through a network. The relationship of client and server may arise by virtue of computer programs running on the respective computers and having a client-server relationship to each other.

One or more features or steps of the disclosed embodiments may be implemented using an API. An API may define one or more parameters that are passed between a calling application and other software code (e.g., an operating system, library routine, function) that provides a service, that provides data, or that performs an operation or a computation.

The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer will employ to access functions supporting the API.

In some implementations, an API call may report to an application the capabilities of a device running the application, such as input capability, output capability, processing capability, power capability, communications capability, etc.

While various embodiments have been described above, it should be understood that they have been presented by way of example and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. For example, other steps may be provided, or steps may be eliminated, from the described flows, and other components may be added to, or removed from, the described systems. Accordingly, other implementations are within the scope of the following claims.

In addition, it should be understood that any figures which highlight the functionality and advantages are presented for example purposes only. The disclosed methodology and system are each sufficiently flexible and configurable such that they may be utilized in ways other than that shown.

Although the term “at least one” may often be used in the specification, claims and drawings, the terms “a”, “an”, “the”, “said”, etc. also signify “at least one” or “the at least one” in the specification, claims and drawings.

Finally, it is the applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

What is claimed is:

1. A device for measuring cervical range of motion comprising:
a frame of a pair of glasses or similar configured to be worn over each ear of a user and on a bridge of a nose of the user, the frame including a positioning clip and laser alignment and optics integrated into a temple of the frame, the positioning clip adjustable between multiple calibration points on the temple in order to ensure a repeatable initial position of the device; and
the laser and optics apparatus to ensure repeatable initial orientation of the device
one or more movement measurement devices integrated into the frame,
the one or more movement measurement devices including one or more sensors, a processor, a memory and a power supply, wherein the memory includes instructions executable by the processor and the processor is configured to:
calibrate the one or more movement measurement devices;
determine, using the one or more movement measurement devices and a laser alignment system, an initial position and orientation for a head or neck of the user;
measure, using the one or more movement measurement devices, a cervical range of motion of the user and a trajectory of motion of the user; and
store the cervical range of motion and the trajectory of motion of the user as movement data.

2. The device of claim 1, wherein the cervical range of motion includes six components of cervical motion, and the one or more sensors includes at least one accelerometer, gyroscope, and magnetometer configured to measure each component of the cervical range of motion.

3. The device of claim 2, wherein the six components of the cervical range of motion include left rotation, right rotation, forward flexion, backward flexion, right flexion, and left flexion.

4. The device of claim 1, wherein the processor is further configured to record the movement data in a database; and
compare the movement data to historical movement data included in the database to monitor a recovery process of the user.

5. The device of claim 4, further comprising a communications interference in communication with the motion sensing device,
wherein the communications interface connects to a remote client device; and
wherein the processor is further configured to transmit the movement data to the remote client device via the communications interface.

6. A system for measuring cervical range of motion comprising:
a frame of a pair of glasses configured to be worn over each ear of a user and on a bridge of a nose of the user; and
a movement measuring device integrated into the frame,
the movement measurement device including multiple sensors, a processor, a memory and a power supply, wherein the memory includes instructions executable by the processor and the processor is configured to:
calibrate the movement measuring device;
determine, using the movement measurement device and a laser alignment system, an initial position and orientation of the heat of each user;
measure, using the movement measurement device, a cervical range of motion for the user and a trajectory of motion for the user; and
store the cervical range of motion and the trajectory of motion as movement data; and
a remote client device connected to the movement measurement device, the remote client device including a processor and a memory connected to a display,
wherein the memory of the remote client device includes instructions executable by the processor of the remote client device and the processor of the remote client device is configured to:
receive the movement data from the motion measurement device;
generate a graphical user interface (GUI) incorporating the movement data; and
present the GUI on the display.

7. The system of claim 6, wherein the GUI includes a chart illustrating the movement data relative to one or more other pieces of movement data.

8. The system of claim 6, wherein the cervical range of motion includes six components of cervical motion, and the movement measurement device includes an accelerometer, gyroscope and magnetometer configured to measure each component of cervical motion.

9. The system of claim 8, wherein the six components of the cervical range of motion include left rotation, right rotation, forward flexion, backward flexion, right flexion, and left flexion.

10. A method for detecting cervical range of motion comprising:
initiating a movement measurement session on a movement measurement device configured to be worn on a portion of a head of a user;
calibrating the movement measurement device;
determining an initial head position and orientation for each user;
measuring a cervical range of motion of the user and a trajectory of motion of the user by:
using the movement measurement device to track a head position of the user relative to the initial head position and orientation as the user performs a head and neck movement; and
storing the cervical range of motion as movement data.

11. The method of claim 10, further comprising comparing each patient's movement data with cervical range of motion data (gender, age, etc. adjusted) from (a) the healthy general population, (b) similarly impaired population and their recovery profiles, and (c) the patient's own records, if and
based on the comparison, estimating a recovery time for the head and neck injury suffered by the user.

12. The method of claim 10, wherein the cervical range of motion includes six components of the cervical range of motion and the movement measurement device includes an accelerometer, gyroscope, and magnetometer configured to measure six components of cervical motion.

13. The method of claim 12, wherein the six components of the cervical range of motion include left rotation, right rotation, forward flexion, backward flexion, right flexion, and left flexion.

14. The method of claim 13, further comprising analyzing the movement data to compare a number of degrees of motion for each component of the cervical range of motion; and
based on the comparison, determining components of the cervical range of motion having an impaired range of motion.

15. The method of claim 14, further comprising analyzing the movement data to prescribe a new physical therapy treatment for the component of the cervical range of motion having the lowest number of degrees of motion.

16. The method of claim 10, further comprising transferring the movement data to a remote client device during a telemedicine consultation with a healthcare provider, wherein the health care provider provides instructions to the user on how the user should sit and move during the movement measurement session.

17. The method of claim 10, further comprising generating a graphical user interface (GUI) including a chart illustrating the movement data; and
displaying on a display of the remote client device, the graphical user interface.

18. The method of claim 10, wherein the calibrating the motion sensing device comprises configuring the movement measurement device to measure degrees of motion within a range of tolerance, for example, in any desired planes including Cartesian planes

19. The method of claim 10, further comprising aligning the head of the user at the initial position and orientation so that the initial position is consistent across multiple movement measurement sessions, wherein the initial head position comprises the head of the user centered over the neck of the user and in a consistent forward-looking position.

20. The method of claim 10, wherein the head and neck movement are selected from a routine of head and neck movements that ensure the head of the user is moved in one direction.
